(19) United States
(12) Patent Application Publication
(10) Pub. No.: US 2019/0251347 A1
(43) Pub. Date: Aug. 15, 2019

(54) OBJECT CHARACTERIZATION AND AUTHENTICATION

(71) Applicant: Sterling Jewelers, Inc., Akron, OH (US)

(72) Inventors: Andrew J. Timpone, Richfield, OH (US); Tyson A. Corvin, Pepper Pike, OH (US); Jason Edleman, Strongsville, OH (US); John Liebler, Medina, OH (US); Melissa Suzanne Scott, Wadsworth, OH (US); Robert Allen Ruffner, Streetsboro, OH (US)

(73) Assignee: Sterling Jewelers, Inc., Akron, OH (US)

(21) Appl. No.: 16/342,916

(22) PCT Filed: Oct. 19, 2017
(86) PCT No.: PCT/US17/57458

§ 371 Date: Apr. 17, 2019

Related U.S. Application Data
(60) Provisional application No. 62/414,256, filed on Oct. 28, 2016.

(51) Int. Cl. G06K 9/00; G01N 33/38; G01N 21/87

(52) U.S. Cl. CPC G06K 9/00577; G01N 33/381; G06K 2009/0059; G06K 9/00671; G01N 21/87

(57) ABSTRACT

A method for object characterization and authentication is described. In one embodiment, the method may include establishing, over a communication network, a connection between a network accessible image capturing device and a remote computing device, capturing a first image of an object using the microscope before performing an action in relation to the object, identifying a distinguishing feature of the object on the first image of the object, capturing a second image of the object using the microscope after performing the action in relation to the object, and identifying the distinguishing feature of the object on the second image of the object. In one embodiment, the image capturing device may include, or be associated with, a microscope.

### CROSS-REFERENCE TO RELATED APPLICATIONS

This application claims priority to U.S. Provisional Patent Application No. 62/414,256 filed Oct. 28, 2016, and titled “Object Characterization and Authentication,” the disclosure of which is hereby incorporated in its entirety by this reference.

### BACKGROUND

In various situations, an owner of an object may leave the object in the care of a third party. For example, the owner may leave the object temporarily with a third party to allow the third party to perform a service in relation to the object (e.g., repair or cleaning of the object). In some cases, when the owner reclaims the object from the third party, the owner may seek assurances from the third party that the object he/she left with the third party is the same object that the owner left with the third party.

### DISCLOSURE OF THE INVENTION

According to at least one embodiment, a method for object characterization and authentication is described. In one embodiment, the method may include establishing, over a communication network, a connection between a network accessible microscope and a remote computing device, capturing a first image of an object using the microscope before performing an action in relation to the object, identifying one or more distinguishing features of the object on the first image of the object, capturing a second image of the object using the microscope after performing the action in relation to the object, and identifying the one or more distinguishing features of the object on the second image of the object.

In some cases, the microscope (or an image capture device associated with the microscope) may include one or more web services. In some cases, the one or more web services may include a device user interface accessible by the remote computing device via the connection. In some embodiments, the capturing of the first image may include sending a first image capture command from the remote computing device to the microscope via the device user interface. In some cases, the capturing of the second image may include sending a second image capture command from the remote computing device to the microscope via the device user interface.

In some embodiments, the method may include accessing at least one of the first and second images of the object on the remote computing device via a device user interface. In some embodiments, the method may include marking, via the device user interface, the distinguishing feature of the object on the first image of the object. In some embodiment, the method may include an automated process. In some cases, the automated process may include any combination of software, firmware, and hardware configured for detection, marking, and communication of distinguishing features in objects without or with limited human intervention. For example, the automated process may include identifying the distinguishing feature, identifying the location of the distinguishing feature on the first image of the object, and automatically adding a marking or indicator to the first image of the object to indicate that the distinguishing feature has been identified and to indicate the location of the identified distinguishing feature on the first image of the object. In some cases, the method may include annotating owner information and/or object information onto the first image of the object. In some embodiments, the owner and/or object information may be annotated on the first image of the object automatically as part of the automated process. In some embodiments, the method may include generating a first communication that includes the first captured image of the object with the distinguishing feature marked. In some cases, the automated process may include generating the first communication. In some embodiments, the method may include marking, via the device user interface, the distinguishing feature of the object on the second image of the object. Additionally, or alternatively, the automated process may include marking of the distinguishing feature on the second image of the object.

In some embodiments, the method may include generating a second communication that includes both the first captured image of the object with the distinguishing feature marked, and the second captured image of the object with the distinguishing feature marked. In some cases, the object may include a gemstone. In some embodiments, the distinguishing feature of the object may include at least one of a type of object, measurement of the object, a diameter of the object, an inclusion of the object, a rating or grading of the object, and an identifier on the object such as a laser inscription. In some cases, the object may include a top side, the first and second images capturing a view of the top side of the object.

A computing device configured for object characterization and authentication is also described. The computing device may include a processor and memory in electronic communication with the processor. The memory may store computer executable instructions that when executed by the processor cause the processor to perform the steps of establishing, over a communication network, a connection between a network accessible microscope and a remote computing device, capturing a first image of an object using the microscope before performing an action in relation to the object, identifying a distinguishing feature of the object on the first image of the object, capturing a second image of the object using the microscope after performing the action in relation to the object, and identifying the distinguishing feature of the object on the second image of the object.

A non-transitory computer-readable storage medium storing computer executable instructions is also described. When the instructions are executed by a processor, the execution of the instructions may cause the processor to perform the steps of establishing, over a communication network, a connection between a network accessible microscope (or related device) and a remote computing device, capturing a first image of an object using the microscope before performing an action in relation to the object, identifying a distinguishing feature of the object on the first image of the object, capturing a second image of the object using the microscope after performing the action in relation to the object, and identifying the distinguishing feature of the object on the second image of the object.

Features from any of the above-mentioned embodiments may be used in combination with one another in accordance with the general principles described herein. These and other embodiments, features, and advantages will be more fully understood upon reading the following detailed description in conjunction with the accompanying drawings and claims.

### BRIEF DESCRIPTION OF THE DRAWINGS

The accompanying drawings illustrate a number of exemplary embodiments and are a part of the specification. Together with the following description, these drawings demonstrate and explain various principles of the instant disclosure.

FIG. 1A illustrates one embodiment of an environment in which the present systems and methods may be implemented;

FIG. 1B is a block diagram illustrating an embodiment of an environment, such as that shown in FIG. 1A, in which the present systems and methods may be implemented;

FIG. 1C is a block diagram illustrating one embodiment of an environment in which the present systems and methods may be implemented;

FIG. 2 is a block diagram illustrating one example of an object authentication module;

FIG. 3 is a block diagram illustrating one example of an environment for object characterization and authentication;

FIG. 4 is a block diagram illustrating one example of an environment for object characterization and authentication;

FIG. 5 is a block diagram illustrating one example of an environment for object characterization and authentication;

FIG. 6 is a flow diagram illustrating one embodiment of a method for object characterization and authentication;

FIG. 7 is a flow diagram illustrating one embodiment of a method for object characterization and authentication;

FIG. 8 is a flow diagram illustrating one embodiment of a method for object characterization and authentication; and

FIG. 9 depicts a block diagram of a computer system suitable for implementing the present systems and methods.

While the embodiments described herein are susceptible to various modifications and alternative forms, specific embodiments have been shown by way of example in the drawings and will be described in detail herein. However, the exemplary embodiments described herein are not intended to be limited to the particular forms disclosed. Rather, the instant disclosure covers all modifications, equivalents, and alternatives falling within the scope of the appended claims.

### BEST MODE(S) FOR CARRYING OUT THE INVENTION

The systems and methods described herein relate to object characterization and authentication. More specifically, the systems and methods described herein relate to object characterization and authentication in relation to an object an owner leaves in the care of a third party. In some cases, an owner may leave an object temporarily in the care of a third party to enable the third party to perform a service in relation to the object such as perform maintenance on the object, clean the object, repair the object, etc. In some cases, when reclaiming the object from the third party, the owner may seek assurances from the third party that the object he/she left with the third party is the same item the owner left with the third party. Accordingly, the third party may characterize the object and then authenticate the object when the owner returns for the object. For example, the third party may capture one or more images of an item to identify distinguishing characteristics of the item.

In one embodiment, the third party may share the distinguishing characteristics identified in the one or more images of the item with the owner, along with other information as owner name, address, and owner signature. For example, the owner may provide his/her signature to indicate that the owner verifies the image of the item is an image of the owner's item. When the owner returns for the item, the third party may capture one or more new images of the item (e.g., after the service has been performed). The third party may then show the owner a comparison of the first set of images of the item when the item was left in the care of the third party with the second set of images of the item when the owner returned to pick up the item, enabling the owner to confirm that the item the owner left is the same item the owner is reclaiming. The third party may again take the signature of the owner indicating the owner verifies that the first and second images are images of the owner's item and the item returned to the owner is the item the owner left with the third party.

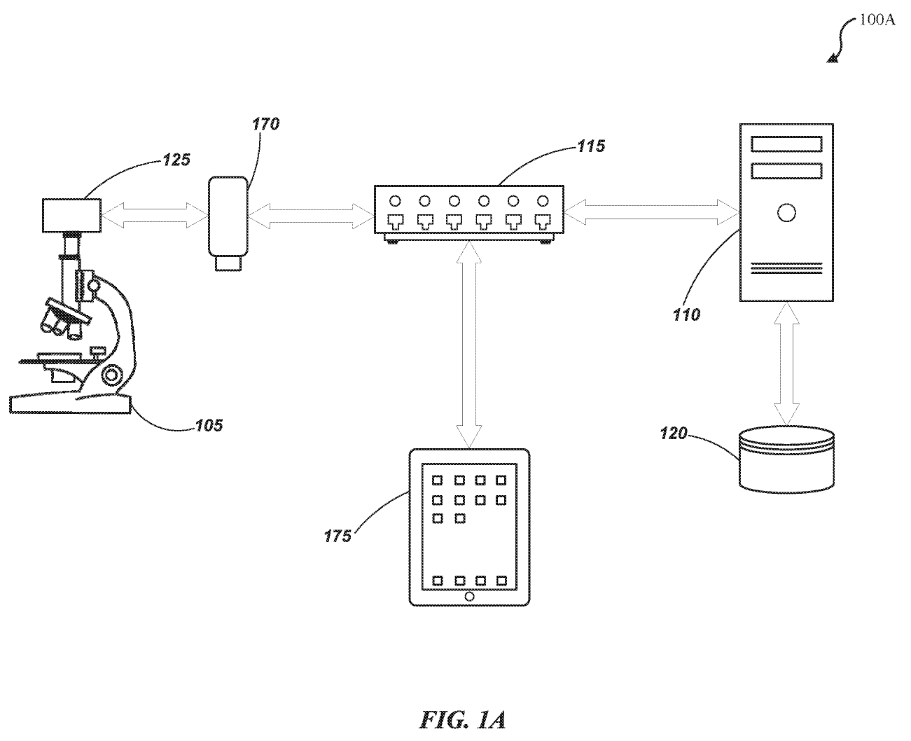

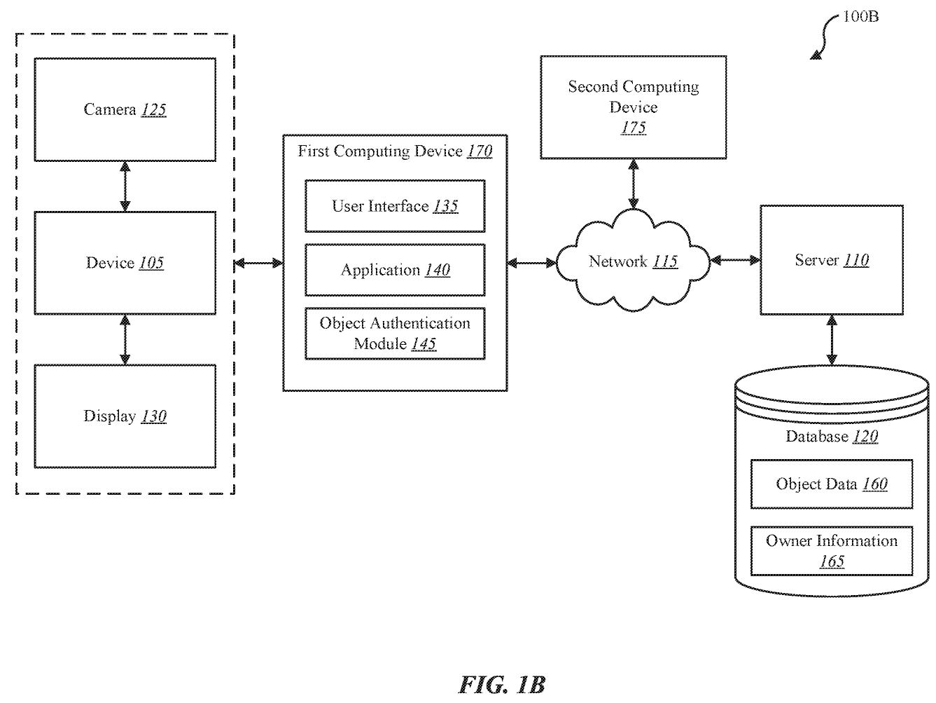

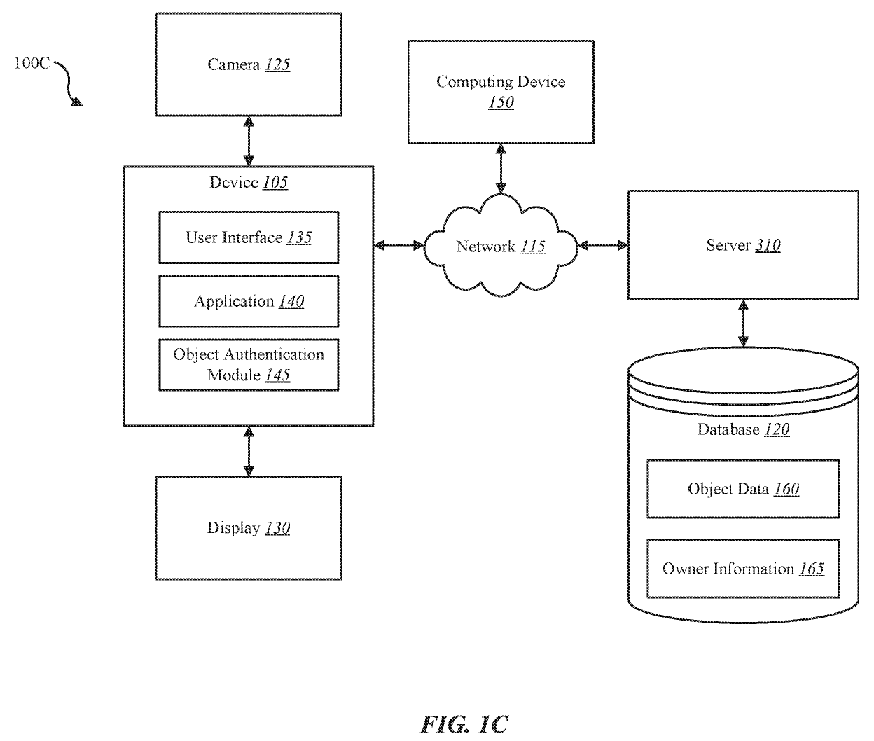

FIGS. 1A and 1B illustrate one embodiment of an environment 100A and 100B, respectively, in which the present systems and methods may be implemented. In some embodiments, the systems and methods described herein may be performed on a device (e.g., device 105). As depicted, the environment 100A may include a device 105, a server 110, a camera 125, a display 130, a first computing device 170, a second computing device 175, and a network 115 that allows device 105, server 110, first computing device 170, and second computing device 175 to communicate with one another.

Examples of the device 105 may include any combination of microscopes, microscope cameras (e.g., camera 125), microscope network adapters, microscope displays (e.g., display 130), mobile devices, smart phones, personal computing devices, computers, laptops, desktops, servers, media content set top boxes, digital video recorders (DVRs), or any combination thereof. In some cases, device 105 may display images of a microscope via display 130. Likewise, in some cases, device 105 may capture images of a microscope via camera 125.

Examples of computing device 175 may include any combination of a mobile computing device, a laptop, a desktop, a server, a media set top box, or any combination thereof. Examples of server 110 may include any combination of a data server, a cloud server, a server associated with an automation service provider, proxy server, mail server, web server, application server, database server, communications server, file server, home server, mobile server, name server, or any combination thereof.

As depicted, first computing device 170 may include user interface 135, application 140, and object authentication module 145. In one embodiment, first computing device 170 may connect to device 105, camera 125, and/or display 130. For example, first computing device 170 may connect to a port such as a universal serial bus (USB) port of device 105, camera 125, or display 130. In some embodiments, first computing device 170 may connect to at least one of device 105, camera 125, and display 130 over a wireless connection.

In some configurations, the first computing device 170 may include a user interface 135, application 140, and object authentication module 145. Although the components of the first computing device 170 are depicted as being internal to the first computing device 170, it is understood that one or more of the components may be external to first computing device 170 and connect to first computing device 170 through wired and/or wireless connections. Application 140 may include one or more web applications. In some cases, application 140 may implement one or more representational state transfer (REST) or RESTful protocols and/or web services. In some cases, application 140 may include one or more hypertext markup language (HTML) protocols such as HTMLS protocols.

In some embodiments, first computing device 170 may enable a user to interface with device 105, camera 125, and/or display 130. For example, first computing device 170 may enable a user to connect to and control one or more aspects of device 105, camera 125, and/or display 130 such as invoke an action in relation to device 105, camera 125, and/or display 130. As one example, device 105 may include a microscope and first computing device 170 may enable a user to invoke camera 125 to capture an image in view of the microscope. In one embodiment, first computing device 170, in conjunction with object authentication module 145, may enable a user on second computing device 175 to connect to and control one or more aspects of device 105, camera 125, and/or display 130 over network 115. For example, object authentication module 142 of first computing device 170 may receive a command sent by second computing device 175 over network 115 and relay the command to at least one of device 105, camera 125, and display 130 to invoke an action such as invoking camera 125 to capture an image in relation to device 105. Further details regarding the object authentication module 145 are discussed below.

In some embodiments, first computing device 170 may communicate with server 110 via network 115. Examples of network 115 may include any combination of cloud networks, local area networks (LAN), wide area networks (WAN), virtual private networks (VPN), wireless networks (using 802.11, for example), cellular networks (using 3G and/or LTE, for example), etc. In some configurations, the network 115 may include the Internet. It is noted that in some embodiments, the first computing device 170 may not include an object authentication module 145. For example, first computing device 170 may include application 140 that allows first computing device 170 to interface with second computing device 175 and/or server 110 via an object authentication module 145 located on another device such as second computing device 175 and/or server 110.

In some embodiments, first computing device 170 and server 110 may include an object authentication module 145 where at least a portion of the functions of object authentication module 145 are performed separately and/or concurrently on first computing device 170, and/or server 110. Likewise, in some embodiments, a user may access the functions of first computing device 170 (directly or through first computing device 170 via object authentication module 145) from second computing device 175. For example, in some embodiments, second computing device 175 includes a mobile application that interfaces with one or more functions of first computing device 170, object authentication module 145, and/or server 110.

In some embodiments, server 110 may be coupled to database 120. Database 120 may be internal or external to the server 110. In one example, device 105 may be coupled directly to database 120, database 120 being internal or external to device 105. Database 120 may include object data 160 and owner information 165. For example, device 105 may access object data 160 in database 120 over network 115 via server 110. Object data 160 may include data regarding an object such as a type of object, a measurement of the object, a diameter of the object, an inclusion of the object, a rating or grading of the object, an identifier on the object, or any combination thereof. In some cases, the type of object may include gemstone and/or type of gemstone such as pearl, diamond, emerald, ruby, sapphire, etc. In some cases, the type of object may specify jewelry and/or type of jewelry such as ring, earring, bracelet, necklace, loose gemstone, etc. Owner information 165 may include data related to an owner of the object such as name, address, phone number, email address, owner signature, credit card information, etc.

Object authentication module 145 may enable capturing images of an object, characterizing a distinguishing feature of the object from at least a first captured image of the object, and authenticating the object by characterizing the same distinguishing feature of the object from at least a second, subsequent captured image of the object. In some embodiments, object authentication module 145 may be configured to perform the systems and methods described herein in conjunction with user interface 135 and application 140. User interface 135 may enable a user to interact with, control, and/or program one or more functions of object authentication module 145. Further details regarding the object authentication module 145 are discussed below.

FIG. 1C is a block diagram illustrating another embodiment of an environment 100C in which the present systems and methods may be implemented. In some embodiments, the systems and methods described herein may be performed on a device (e.g., device 105). As depicted, the environment 100C may include a device 105, a server 110, a camera 125, a display 130, a computing device 150, and a network 115 that allows device 105, server 110, and computing device 150 to communicate with one another. Examples of computing device 150 may include those described with regard to computing device 175 hereinabove.

In some configurations, the device 105 may include a user interface 135, application 140, and object authentication module 145. Although the components of the device 105 are depicted as being internal to the device 105, it is understood that one or more of the components may be external to the device 105 and connect to device 105 through wired and/or wireless connections. Application 140 may include one or more web applications. In some cases, application 140 may implement one or more representational state transfer (REST) or RESTful protocols and/or web services. In some cases, application 140 may include one or more hypertext markup language (HTML) protocols such as HTMLS protocols. In some embodiments, one or more elements of application 140 may be installed on computing device 150 in order to allow a user of computing device 150 to interface with a function of device 105, object authentication module 145, and/or server 110.

In some embodiments, device 105 may communicate with server 110 via network 115. Examples of network 115 may include any combination of cloud networks, local area networks (LAN), wide area networks (WAN), virtual private networks (VPN), wireless networks (using 802.11, for example), cellular networks (using 3G and/or LTE, for example), etc. In some configurations, the network 115 may include the Internet. It is noted that in some embodiments, the device 105 may not include an object authentication module 145. For example, device 105 may include application 140 that allows device 105 to interface with computing device 150 and/or server 110 via an object authentication module 145 located on another device such as computing device 150 and/or server 110.

In some embodiments, device 105, and server 110 may include an object authentication module 145 where at least a portion of the functions of object authentication module 145 are performed separately and/or concurrently on device 105, and/or server 110. Likewise, in some embodiments, a user may access the functions of device 105 (directly or through device 105 via object authentication module 145) from computing device 150. For example, in some embodiments, computing device 150 includes a mobile application that interfaces with one or more functions of device 105, object authentication module 145, and/or server 110.

In some embodiments, server 110 may be coupled to database 120. Database 120 may be internal or external to the server 110. In one example, device 105 may be coupled directly to database 120, database 120 being internal or external to device 105. Database 120 may include object data 160 and owner information 165. For example, device 105 may access object data 160 in database 120 over network 115 via server 110. Object data 160 may include data regarding an object such as a type of object, a measurement of the object, a diameter of the object, an inclusion of the object, a rating or grading of the object, an identifier on the object, or any combination thereof. In some cases, the type of object may include gemstone and/or type of gemstone such as pearl, diamond, emerald, ruby, sapphire, etc. In some cases, the type of object may specify jewelry and/or type of jewelry such as ring, earring, bracelet, necklace, loose gemstone, etc. Owner information 165 may include data related to an owner of the object such as name, address, phone number, email address, owner signature, credit card information, etc.

Object authentication module 145 may enable capturing images of an object, characterizing a distinguishing feature of the object from at least a first captured image of the object, and authenticating the object by characterizing the same distinguishing feature of the object from at least a second, subsequent captured image of the object. In some embodiments, object authentication module 145 may be configured to perform the systems and methods described herein in conjunction with user interface 135 and application 140. User interface 135 may enable a user to interact with, control, and/or program one or more functions of object authentication module 145. Further details regarding the object authentication module 145 are discussed below.

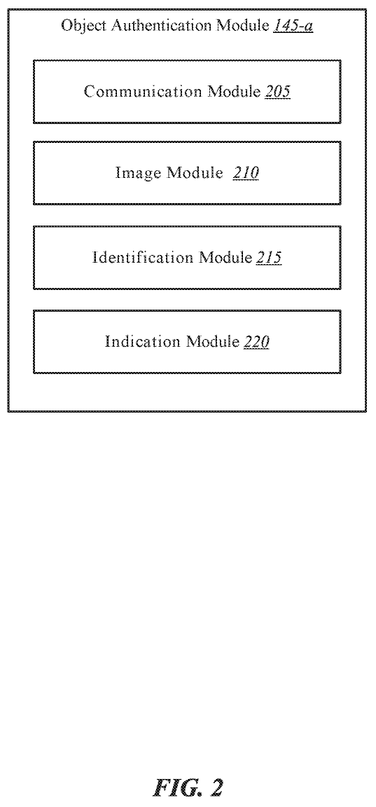

FIG. 2 is a block diagram illustrating one example of an object authentication module 145-a. Object authentication module 145-a may be one example of object authentication module 145 depicted in FIGS. 1A, 1B, and/or 1C. As depicted, object authentication module 145-a may include communication module 205, image module 210, identification module 215, and indication module 220.

In one embodiment, communication module 205 may be configured to establish a connection between a microscope (or an associated image capturing device such as a camera) and a remote computing device. Although a microscope is described herein as a device for capturing images, other types of devices may be used in place of or in addition to a microscope. For example, any device that provides an enlarged and/or detailed an/or close up view of an object (e.g., a gemstone) may be used. In some cases, the microscope (or other image capturing device) may be network accessible. In some embodiments, communication module 205 may be configured to establish the connection between the microscope (or other image capturing device) and a remote computing device over a communication network such as a transmission control protocol (TCP) and/or internet protocol (IP) network. In some cases, the connection over the communication network may include wired and/or wireless network connections. In some cases, the microscope (or other image capturing device) may include one or more web services. In some embodiments, the one or more web services may include a device user interface (e.g., user interface 135 of FIGS. 1A, 1B, and/or 1C). The device user interface may make one or more features of the microscope (or other image capturing device) accessible to a remote computing device via a network connection between the remote computing device and the microscope (or other image capturing device).

In some embodiments, image module 210 may be configured to capture a first image of an object. Although reference is made herein to a first image and a second image, it is understood that reference to “first image” may represent one or more first images and that reference to “second image” may represent one or more second images. For example, in one embodiment, reference to “first image” may refer to capturing one or more images of an object at a first time and reference to “second image” may refer to capturing one or more images of the object at a second time, the second time being a time after the first time such as a number of minutes later, one hour later, one day later, one week later, etc.

In some cases, the object may include a top side. In one embodiment, the image module 210 may capture a view of the top side of the object in the first image of the object. In one embodiment, the capturing of the first image may include communication module 205 sending a first image capture command from the remote computing device to the microscope via the device user interface. For example, the device user interface may include options displayed to a user of the remote computing device. The options of the device user interface may include, for example, view an object via the microscope, capture an image of an object via the microscope, access the image of the object, create a copy of the image of the object, add (or modify) an indicator to the image of the object that indicates a distinguishing feature of the object, link owner information with the image of the object, add (or modify) an annotation to the image of the object such as an annotation that includes owner information and/or features of the object, “lock” the data (including annotations) associated with an image, and send the image of the object in a message (by email and/or text message, for example), etc.

In some cases, image module 210 may capture the first image of the object in conjunction with a microscope. In some cases, image module 210 may capture the first image in conjunction with a camera associated with the microscope. In some embodiments, image module 210 may be configured to capture the first image of the object before an action or service is performed in relation to the object. For example, an owner of the object may leave the object in the care of a third party. In some cases, the third party may perform an action or service in relation to the object such as, for example, cleaning and/or repair the object.

In some embodiments, identification module 215 may be configured to identify a distinguishing feature of the object on the first image of the object. In some cases, the distinguishing feature of the object may include a measurement of the object, a diameter of the object, an inclusion of the object, a rating or grading of the object, an identifier on the object, or any combination thereof.

In one embodiment, identification module 215 may automatically detect a distinguishing feature of an object without human input. For example, identification module 215 may implement any combination of software, firmware, and hardware configured to detect features of an object, mark or indicate detected features of the object on an image of the object, and/or communicate the marked image of the object via an automated process. In some cases, identification module 215 may include specialized software, firmware, and/or hardware configured to detect the features without human input. For example, identification module 215 may include an algorithm configured for detecting features of an object. As one example, identification module 215 may implement one or more facial recognition algorithms. In some cases, identification module 215 may implement a facial recognition algorithm to detect a feature of an object. In some embodiments, identification module 215 may implement a facial recognition algorithm that is tuned, modified and/or specialized for detecting features of an object such as features of a gemstone. When using such automation features, a user may override, modify, remove or further annotate a feature that was automatically identified and/or characterized by the identification module. Once all features have been positively identified (whether through an automated process, by human identification, or both) and finalized with regard to annotation, a user may use the user interface to submit the images as a final version, thereby “locking” the identification of features and associated annotations. At this point, the images (along with any identifiers and annotations) become read-only and may not be further altered.

In some embodiments, the object may include a gemstone. An inclusion identified by identification module 215 may include a body or particle recognizably distinct from the substance in which it is embedded. An inclusion of a mineral or gemstone may include any material that is trapped within the mineral or gemstone during its formation. For example, an inclusion in an emerald may include a cavity or particle recognizably distinct from the substance of the emerald. In some cases, the identifier of the object may be inscribed or laser-etched into the object such as a laser inscription identifier inscribed into the object. Thus, the distinguishing feature may include a gemstone cut, gemstone color, gemstone clarity, gemstone carat weight, or any combination thereof. In some embodiments, the gemstone may be tested in conjunction with identification module 215 to verify whether it is an authentic gemstone such as a diamond.

In some embodiments, image module 210 may be configured to capture a second image of the object using the microscope after performing the action in relation to the object. In some embodiments, the capturing of the second image may include communication module 205 sending a second image capture command from the remote computing device to the microscope via the device user interface. In one embodiment, the image module 210 may capture a view of the top side of the object in the second image of the object. In some embodiments, identification module 215 may be configured to identify the distinguishing feature of the object on the second image of the object.

In some embodiments, communication module 205 may be configured to access at least one of the first and second images of the object on a remote computing device via the device user interface. For example, communication module 205, in conjunction with the device user interface, may enable a remote computing device to access and/or retrieve the first and/or second images of the object over the communication network and connection between the remote computing device and the microscope. In some cases, communication module 205 may transfer a copy of the first and/or second images of the object over the communication network from the microscope (or other image capturing device) to the remote computing device.

In some embodiments, indication module 220 may be configured to indicate the distinguishing feature of the object on the first image of the object. In some cases, indication module 220 may be configured to add a marking or indicator on the first image of the object relative to the identified distinguishing feature. In some cases, indication module 220 may be configured to access the first image of the object via the device user interface and then add the indicator to the first image of the object stored at the microscope. In some embodiments, identification module 215 and indication module 220 may include an automated process to perform the steps of identifying the distinguishing feature, identifying the location of the distinguishing feature on the first image of the object, and automatically adding a marking or indicator to the first image of the object to indicate that the distinguishing feature has been identified and to indicate the location of the identified distinguishing feature on the first image of the object. In some cases, the indication module 220 may automatically annotate information to the first image of the object. For example, indication module 220 may annotate owner information to the first image of the object such as owner name, owner address, etc. In some cases, indication module 220 may annotate information to the first image of the object as part of the automated process.

In some cases, indication module 220 may be configured to create a copy of the first image of the object and add an indicator to the copy of the first image. For example, indication module 220 may be configured to add an indicator (e.g., providing identification of a unique characteristic of the object) to a copy of the first image accessed and/or transferred by the communication module 205 over the connection between the microscope and the remote computing device.

In some cases, communication module 205 may create a copy of the first image of the object and store the copy on the remote computing device. In some cases, indication module 220 may be configured to add an indicator to the copy of the first image stored at the remote computer device. In some cases, communication module 205 may store a copy of the first image of the object in a central storage location such as on a cloud storage system, on a database of a server, on a distributed data service, or any combination thereof.

In some embodiments, communication module 205 may be configured to generate a first communication that includes the first captured image of the object with the distinguishing feature marked. In some cases, communication module 205 may be configured to send the first communication to a recipient associated with the object such as an owner of the object. For example, communication module 205 may be configured to send an email or text message to a recipient regarding the first image of the object. In some cases, the first communication may include information about an owner of the object. In some embodiments, communication module 205 may receive owner information about an owner of the object. In some cases, the owner information may be received in conjunction with the remote computing device.

In some embodiments, the communication module 205 may link the owner information with the first image of the object. In some cases, communication module 205 may link the owner information with the object via the device user interface. In some cases, communication module 205 may receive an electronic signature of the customer as part of the owner information. In one embodiment, the first communication sent to the owner may include the electronic signature of the customer in addition to the owner information, the first captured image of the object, and one or more markings on the first captured image of the object identifying the distinguishing features that uniquely identify the object.

In some cases, indication module 220 may be configured to indicate the distinguishing feature of the object on the second image of the object. In some cases, indication module 220 may be configured to add a marking or indicator on the first image of the object relative to the identified distinguishing feature. In some embodiments, communication module 205 may be configured to generate a second communication that includes both the first captured image of the object with the distinguishing feature marked and the second captured image of the object with the distinguishing feature marked.

In some cases, the second communication may include owner information linked to the first and/or second images of the object and/or distinguishing features of the object, etc. In some cases, the second communication may include a signature from the owner indicating that the owner agrees that the object being returned by a third party to the owner is the same object that the owner left with the third party based on a review of the object and/or a comparison by the owner of the first image to the second image. For example, in some embodiments, communication module 205 may display the first image of the object next to the second image of the object to enable the owner of the object to compare the images of the object in the first and second images and verify based on this comparison that the object returned to the owner is the same that the owner left with the third party.

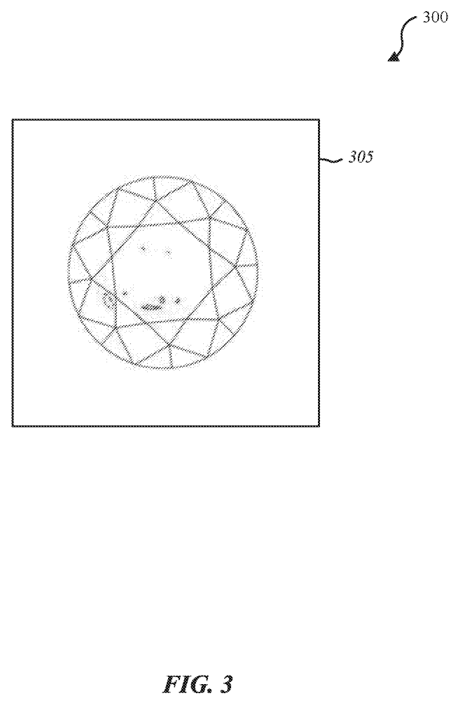

FIG. 3 illustrates one example of an environment 300 for object characterization and authentication. As depicted, environment 300 may include a first image of an object 305. In one example, as shown, the first image of the object 305 may include an image of a gemstone. In some embodiments, the environment 300 may include a microscope equipped with a camera or other image capturing device for capturing images of objects placed in view of the microscope lens. In some cases, environment 300 may include, or be associated with, a storage device for storing images captured by the microscope such as the first image of the object 305 shown in FIG. 3. In some embodiments, environment 300 may include, or be associated with, a communication transceiver communicatively connected to the microscope (or other image capturing device) and configured for transmitting and/or receiving data over a connection between the microscope (or other device) and a remote computing system.

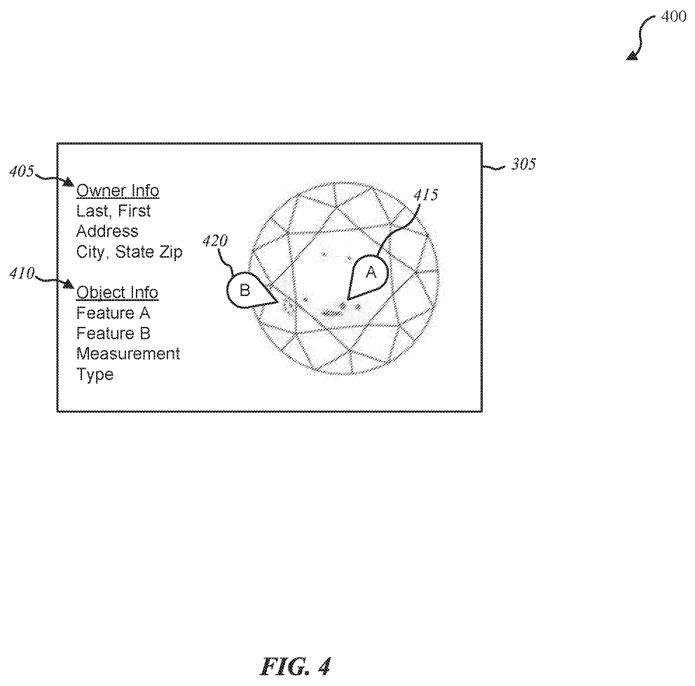

FIG. 4 illustrates one example of an environment 400 for object characterization and authentication. In some cases, environment 400 may be one example of environment 300 of FIG. 3. As depicted, environment 400 may include the first image of the object 305. In some embodiments, the first image of the object 305 may include owner information 405, object information 410, first marker 415, and second marker 420. In some cases, the owner information 405, object information 410, first marker 415, and/or second marker 420 may be appended to the first image of the object 305.

In some embodiments, the owner information 405 and/or object information 410 may be annotated into fields provided by a user interface of the device (e.g., user interface 135 of FIGS. 1A, 1B, and/or 1C). In some cases, pre-configured fields may be provided in relation to the image in which the owner information may be added such as name, address, telephone, email, credit card information, etc.

In some embodiments, the owner information 405 and/or object information 410 entered in the device user interface may be linked to the first image of the object 305. In one embodiment, a file may be generated that links the first image of the object 305 to the owner information 405 and/or object information 410. Additionally, or alternatively, the first image of the object 305 as well as the owner information 405 and/or object information 410 may be stored in a database and mutually associated with an identifier. For example, the file names for the first image of the object 305 as well as the owner information 405 and/or object information 410 may include a common identifier that links the files to one another. In some cases, the owner information 405 and/or object information 410 entered in the device user interface may be added onto the first image of the object 305. For example, the owner information may be appended to the image and/or annotated onto the image.

In one embodiment, first marker 415 may indicate a first identified distinguishing feature of the object in the first image of the object 305, and second marker 420 may indicate a second identified distinguishing feature of the object in the first image of the object 305. In some embodiments, the first and/or second markers 415 and 420 may be appended to the first image of the object 305 automatically. In some cases, appending at least one of the owner information 405, object information 410, first marker 415, and/or second marker 420 may include an automated process. In some embodiments, the automated process may include any combination of identifying distinguishing features of an object, marking the identified distinguishing features on an image of the object, linking owner and/or object information with the image of the object, appending owner and/or object information onto the image of the object, and/or communicating the marked and/or annotated image of the object in a message such as text or email.

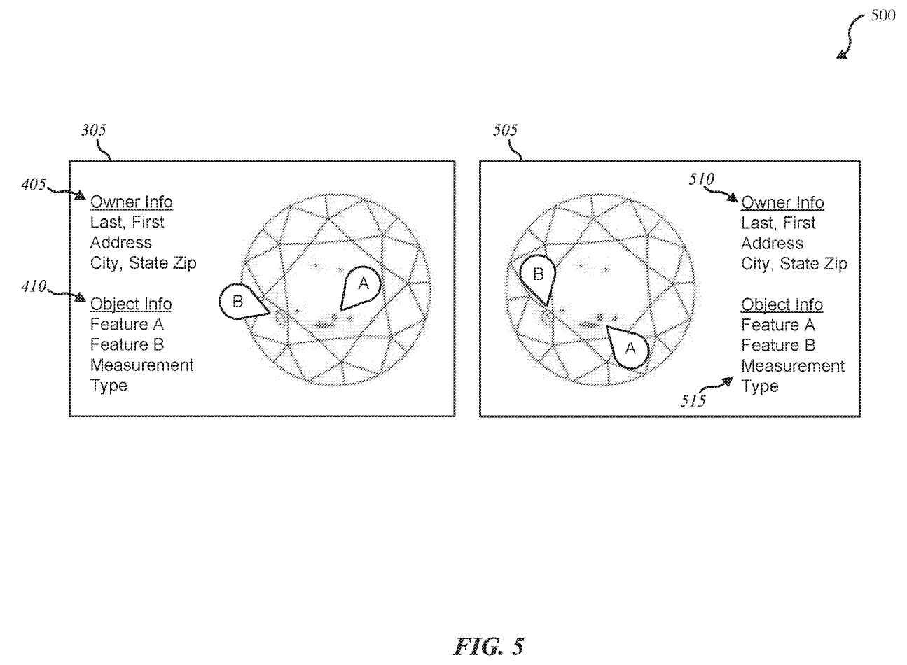

FIG. 5 illustrates one example of an environment 500 for object characterization and authentication. In some cases, environment 500 may be one example of environment 300 of FIG. 3 and/or environment 400 of FIG. 4. As depicted, environment 500 may include the first image of the object 305 and a second image of the object 505.

In one embodiment, the first image of the object 305 and/or the second image of the object 505 may include owner information and/or object information. For example, the first image of the object 305 may include at least one of owner information 405 and object information 410, and/or the second image of the object 505 may include at least one of owner information 510 and object information 515. In some embodiments, the first image of the object 305 and the second image of the object 505 may be shown side by side as depicted. In some cases, the first image of the object 305 and the second image of the object 505 may be shown with one on top and the other on bottom.

In one embodiment, the first image of the object 305 and the second image of the object 505 may be appended into a single image side by side or top over bottom. In one embodiment, the first image of the object 305 and the second image of the object 505 may be sent in a message. For example, the first image of the object 305 and the second image of the object 505 may be sent in an email message and/or an email message. For instance, the first image of the object 305 and the second image of the object 505 may be sent in a message to an owner of the object. In one embodiment, the first image of the object 305 and the second image of the object 505 may be shown relative to one another on a display. In some cases, the owner may view the two images of the object to verify that the object returned to the owner is the same object ha the owner left with a third party.

In some embodiments, the first image of the object 305 and/or second image of the object 505 may be based on live image feeds of the object viewed by the microscope. For example, an image of the object may be viewed live in the presence of the owner when the owner of the object drops the object off with a third party. The live view of the object may be captured by a camera on the microscope. Thus, the first image of the object 305 may be captured as the owner views the object from a live view as seen by the microscope. In some cases, the owner may leave the object in the care of the third party to enable the third party to perform an action in relation to the object such as clean the object or fix the object. The owner may sign that the object in the first image of the object 305 is the same object the owner is leaving in the care of the third party. In some cases, the owner information 405 may include this first signature of the owner.

In one embodiment, the second image of the object 505 may be based on a live feed of the object from the microscope after the third party performs the action (e.g., cleaning, fixing, etc.) on the object. Thus, the owner may see that the object under live view of the microscope is the same object shown in the first image of the object 305. In one embodiment, a signature of the owner may be received that affirms the owner agrees the image of the object in the second image of the object 505 is the same image from the first image of the object 305.

In one embodiment, the second image of the object 505 may be captured from the live image feed of the microscope. For example, the second image of the object 505 may be captured in relation to the owner providing his/her signature based on the live image feed of the object in the microscope shown next to the first image of the object 305. For instance, the second image of the object 505 may include an image of the live feed of the object viewed by the microscope before, while, or after the owner provides his/her signature that the object shown in the live feed is the same object from the first image of the object 305.

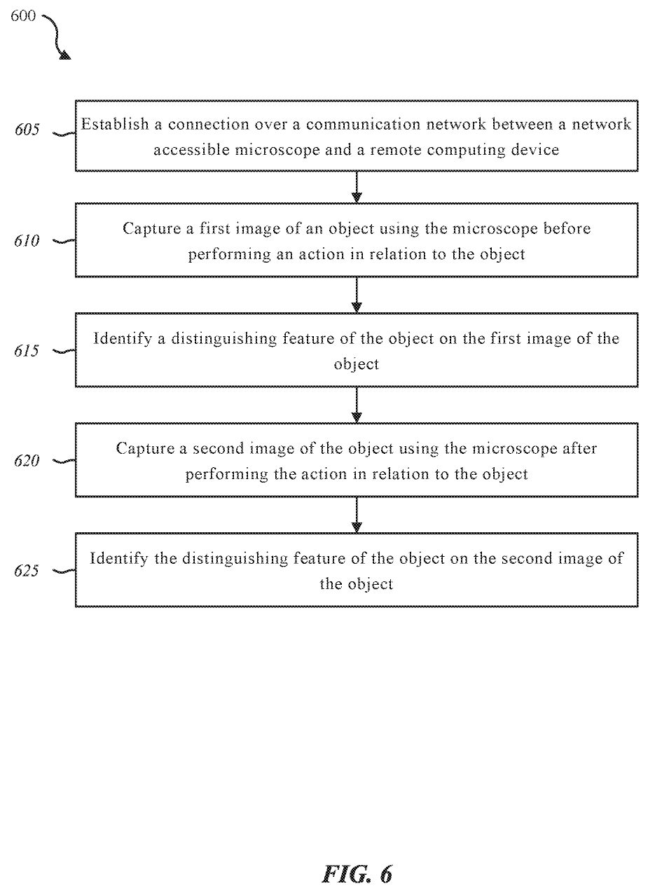

FIG. 6 is a flow diagram illustrating one embodiment of a method 600 for object characterization and authentication. In some configurations, the method 600 may be implemented by the object authentication module 145 illustrated in FIGS. 1 and/or 2. In some configurations, the method 600 may be implemented in conjunction with the application 140 and/or the user interface 135 illustrated in FIGS. 1A, 1B, and/or 1C.

In one embodiment, at block 605, the method 600 may include establishing, over a communication network, a connection between a network accessible microscope (and/or other image capturing device) and a remote computing device. At block 610, the method 600 may include capturing a first image of an object using the microscope (and/or other image capturing device) before performing an action in relation to the object. At block 615, the method 600 may include identifying a distinguishing feature of the object on the first image of the object. At block 620, the method 600 may include capturing a second image of the object using the microscope (and/or other image capturing device) after performing the action in relation to the object. At block 625, the method 600 may include identifying the distinguishing feature of the object on the second image of the object.

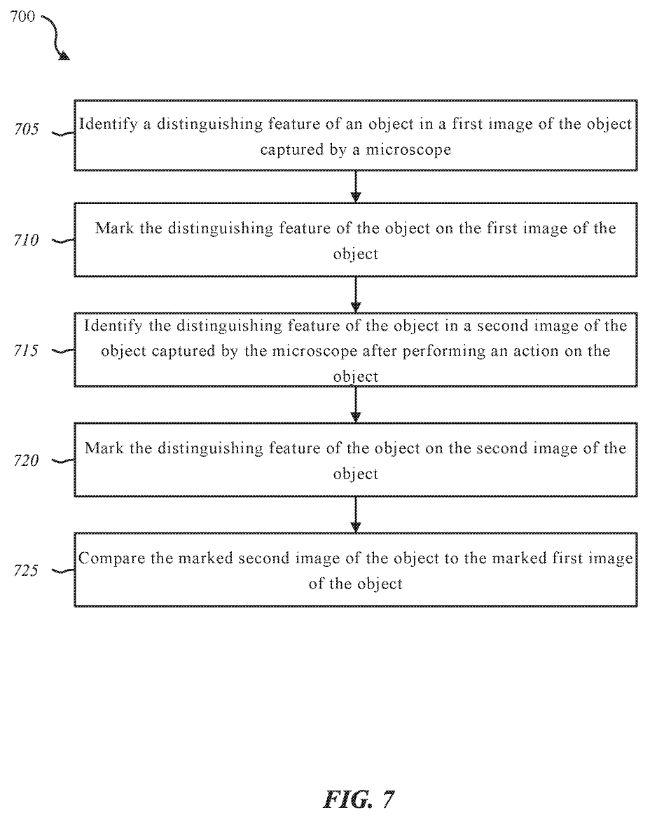

FIG. 7 is a flow diagram illustrating one embodiment of a method 700 for object characterization and authentication. In some configurations, the method 700 may be implemented by the object authentication module 145 illustrated in FIG. 1 or 2. In some configurations, the method 700 may be implemented in conjunction with the application 140 and/or the user interface 135 illustrated in FIGS. 1A, 1B, and/or 1C.

In one embodiment, at block 705, the method 700 may include identifying a distinguishing feature of an object in a first image of the object captured by a microscope (and/or other image capturing device) before performing an action on the object. At block 710, the method 700 may include marking the distinguishing feature of the object on the first image of the object. At block 715, the method 700 may include identifying the distinguishing feature of the object in a second image of the object captured by the microscope (and/or other image capturing device) after performing an action on the object. At block 720, the method 700 may include marking the distinguishing feature of the object on the second image of the object. At block 725, the method 700 may include comparing the marked second image of the object to the marked first image of the object. In some cases, the first and second images of the object may be compared by generating a second communication that includes both the first captured image of the object with the distinguishing feature marked and the second captured image of the object with the distinguishing feature marked.

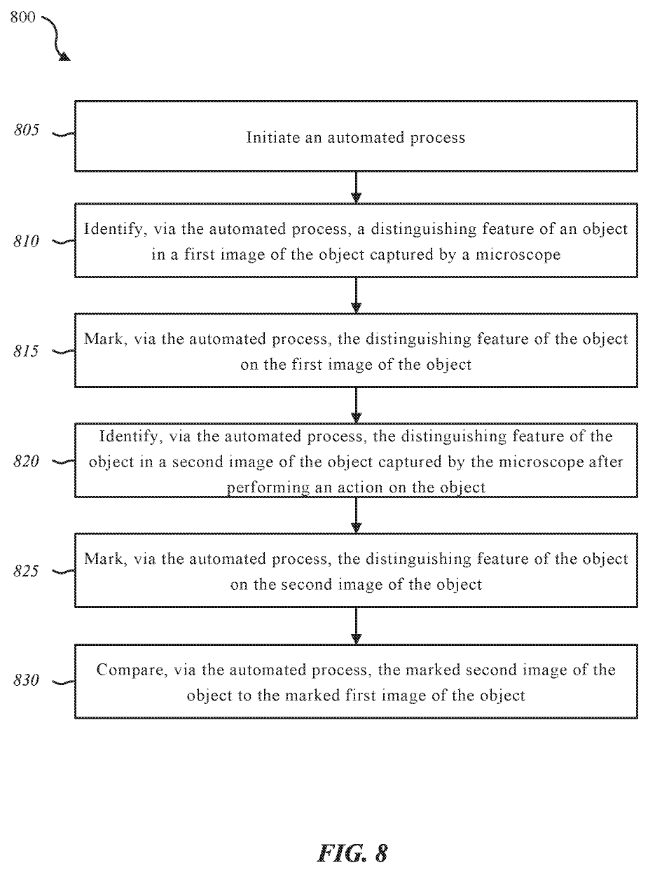

FIG. 8 is a flow diagram illustrating one embodiment of a method 800 for object characterization and authentication. In some configurations, the method 800 may be implemented by the object authentication module 145 illustrated in FIG. 1 or 2. In some configurations, the method 800 may be implemented in conjunction with the application 140 and/or the user interface 135 illustrated in FIGS. 1A, 1B, and/or 1C.

In one embodiment, at block 805, the method 800 may include initiating an automated process. At block 810, the method 800 may include identifying, via the automated process, a distinguishing feature of an object in a first image of the object captured by a microscope (and/or other image capturing device) before performing an action on the object. For example, the automated process may include a facial recognition algorithm or similar feature recognition algorithm that is tuned to detect inclusions in a gemstone. At block 815, the method 800 may include marking, via the automated process, the distinguishing feature of the object on the first image of the object. For example, the automated process may include a software process of identifying the location of the identified distinguishing feature of the object on an image of the object and adding an indicator relative to the identified location. In some cases, the automated process may include generating a first communication that includes an image of the object with one or more indicted distinguishing features. At block 820, the method 800 may include identifying, via the automated process, the distinguishing feature of the object in a second image of the object captured by the microscope (and/or other image capturing device) after performing an action on the object. At block 825, the method 800 may include marking, via the automated process, the distinguishing feature of the object on the second image of the object. The marking may include addition of an annotation such as text, symbols, coloring, etc. At block 830, the method 800 may include comparing, via the automated process, the marked second image of the object to the marked first image of the object. For example, the automated process may include performing image analysis to detect the same identified distinguishing feature in the first and second objects. In some cases, the first and second images of the object may be compared by generating a second communication that includes both the first captured image of the object with the distinguishing feature marked and the second captured image of the object with the distinguishing feature marked.

Although method 800 is directed to a method that includes automatically identifying and labeling distinguishing features in two separate images, the principles discloses with reference to method 800 may be applied in other methods. For example, automatically identifying and labeling distinguishing features may be conducted on a single image rather than on two images. In other examples, the some of the identifying and labeling steps may be performed manually while others are performed automatically. In other examples, three or more images may be analyzed for distinguishing features, compared, etc. In still further examples, the method 800 may include storing and/or saving the images that are marked with distinguishing features.

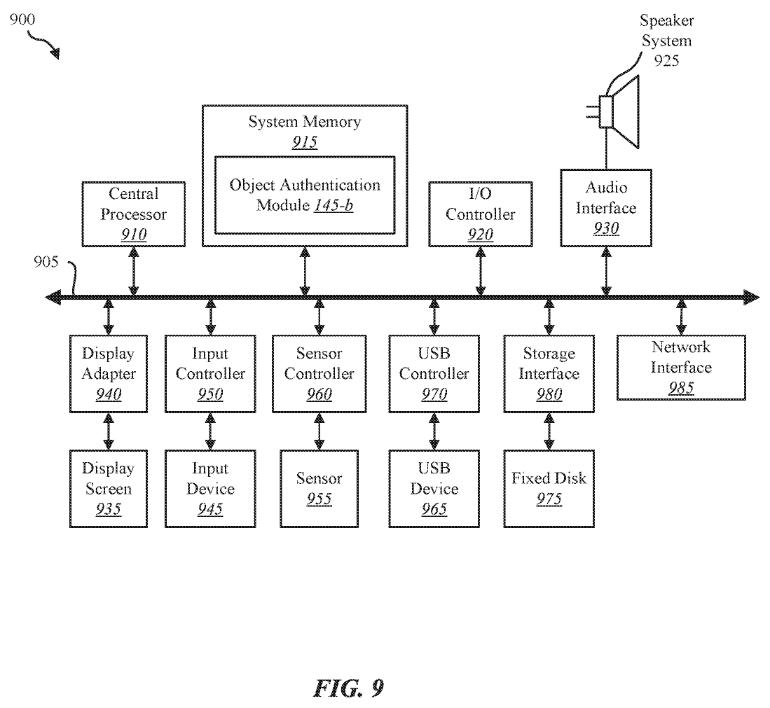

FIG. 9 depicts a block diagram of a computing device 900 suitable for implementing the present systems and methods. The device 900 may be an example of device 105, computing device 150, and/or server 110 illustrated in FIGS. 1A, 1B, and/or 1C. In one configuration, device 900 includes a bus 905 which interconnects major subsystems of device 900, such as a central processor 910, a system memory 915 (typically RAM, but which may also include ROM, flash RAM, or the like), an input/output controller 920, an external audio device, such as a speaker system 925 via an audio output interface 930, an external device, such as a display screen 935 via display adapter 940, an input device 945 (e.g., remote control device interfaced with an input controller 950), multiple USB devices 965 (interfaced with a USB controller 970), and a storage interface 980. Also included are at least one sensor 955 connected to bus 905 through a sensor controller 960 and a network interface 985 (coupled directly to bus 905).

Bus 905 allows data communication between central processor 910 and system memory 915, which may include read-only memory (ROM) or flash memory (neither shown), and random access memory (RAM) (not shown), as previously noted. The RAM is generally the main memory into which the operating system and application programs are loaded. The ROM or flash memory can contain, among other code, the Basic Input-Output system (BIOS) which controls basic hardware operation such as the interaction with peripheral components or devices. For example, the object authentication module 145-b to implement the present systems and methods may be stored within the system memory 915. Applications (e.g., application 140) resident with device 900 are generally stored on and accessed via a non-transitory computer readable medium, such as a hard disk drive (e.g., fixed disk 975) or other storage medium. Additionally, applications can be in the form of electronic signals modulated in accordance with the application and data communication technology when accessed via interface 985.

Storage interface 980, as with the other storage interfaces of device 900, can connect to a standard computer readable medium for storage and/or retrieval of information, such as a fixed disk drive 975. Fixed disk drive 975 may be a part of device 900 or may be separate and accessed through other interface systems. Network interface 985 may provide a direct connection to a remote server via a direct network link to the Internet via a POP (point of presence). Network interface 985 may provide such connection using wireless techniques, including digital cellular telephone connection, Cellular Digital Packet Data (CDPD) connection, digital satellite data connection, or the like. In some embodiments, one or more sensors (e.g., motion sensor, smoke sensor, glass break sensor, door sensor, window sensor, carbon monoxide sensor, and the like) connect to device 900 wirelessly via network interface 985.

Many other devices or subsystems (not shown) may be connected in a similar manner (e.g., entertainment system, computing device, remote cameras, wireless key fob, wall mounted user interface device, cell radio module, battery, alarm siren, door lock, lighting system, thermostat, home appliance monitor, utility equipment monitor, and so on). Conversely, all of the devices shown in FIG. 9 need not be present to practice the present systems and methods. The devices and subsystems can be inter-connected in different ways from that shown in FIG. 9. The aspect of some operations of a system such as that shown in FIG. 9 are readily known in the art and are not discussed in detail in this application. Code to implement the present disclosure can be stored in a non-transitory computer-readable medium such as one or more of system memory 915 or fixed disk 975. The operating system provided on device 900 may be iOS®, ANDROID®, MS-DOS®, MS-WINDOWS®, OS/2®, UNIX®, LINUX®, or another known operating system.

Moreover, regarding the signals described herein, those skilled in the art will recognize that a signal can be directly transmitted from a first block to a second block, or a signal can be modified (e.g., amplified, attenuated, delayed, latched, buffered, inverted, filtered, or otherwise modified) between the blocks. Although the signals of the above described embodiment are characterized as transmitted from one block to the next, other embodiments of the present systems and methods may include modified signals in place of such directly transmitted signals as long as the informational and/or functional aspect of the signal is transmitted between blocks. To some extent, a signal input at a second block can be conceptualized as a second signal derived from a first signal output from a first block due to physical limitations of the circuitry involved (e.g., there will inevitably be some attenuation and delay). Therefore, as used herein, a second signal derived from a first signal includes the first signal or any modifications to the first signal, whether due to circuit limitations or due to passage through other circuit elements which do not change the informational and/or final functional aspect of the first signal.

The signals associated with system 900 may include wireless communication signals such as radio frequency, electromagnetics, local area network (LAN), wide area network (WAN), virtual private network (VPN), wireless network (using 802.11, for example), cellular network (using 3G and/or LTE, for example), and/or other signals. The network interface 985 may enable one or more of WWAN (GSM, CDMA, and WCDMA), WLAN (including BLUETOOTH® and WiFi), WMAN (Wi-MAX) for mobile communications, antennas for Wireless Personal Area Network (WPAN) applications (including RFID and UWB), etc.

The I/O controller 920 may operate in conjunction with network interface 985 and/or storage interface 980. The network interface 985 may enable system 900 with the ability to communicate with client devices (e.g., device 105 of FIGS. 1A, 1B, and/or 1C), and/or other devices over the network 115 of FIGS. 1A, 1B, and/or 1C. Network interface 985 may provide wired and/or wireless network connections. In some cases, network interface 985 may include an Ethernet adapter or Fibre Channel adapter. Storage interface 980 may enable system 900 to access one or more data storage devices. The one or more data storage devices may include two or more data tiers each. The storage interface 980 may include one or more of an Ethernet adapter, a Fibre Channel adapter, Fibre Channel Protocol (FCP) adapter, a SCSI adapter, and iSCSI protocol adapter.

While the foregoing disclosure sets forth various embodiments using specific block diagrams, flowcharts, and examples, each block diagram component, flowchart step, operation, and/or component described and/or illustrated herein may be implemented, individually and/or collectively, using a wide range of hardware, software, or firmware (or any combination thereof) configurations. In addition, any disclosure of components contained within other components should be considered exemplary in nature since many other architectures can be implemented to achieve the same functionality.

The process parameters and sequence of steps described and/or illustrated herein are given by way of example only and can be varied as desired. For example, while the steps illustrated and/or described herein may be shown or discussed in a particular order, these steps do not necessarily need to be performed in the order illustrated or discussed. The various exemplary methods described and/or illustrated herein may also omit one or more of the steps described or illustrated herein or include additional steps in addition to those disclosed.

Furthermore, while various embodiments have been described and/or illustrated herein in the context of fully functional computing systems, one or more of these exemplary embodiments may be distributed as a program product in a variety of forms, regardless of the particular type of computer-readable media used to actually carry out the distribution. The embodiments disclosed herein may also be implemented using software modules that perform certain tasks. These software modules may include script, batch, or other executable files that may be stored on a computer-readable storage medium or in a computing system. In some embodiments, these software modules may configure a computing system to perform one or more of the exemplary embodiments disclosed herein.

The foregoing description, for purpose of explanation, has been described with reference to specific embodiments. However, the illustrative discussions above are not intended to be exhaustive or to limit the invention to the precise forms disclosed. Many modifications and variations are possible in view of the above teachings. The embodiments were chosen and described in order to best explain the principles of the present systems and methods and their practical applications, to thereby enable others skilled in the art to best utilize the present systems and methods and various embodiments with various modifications as may be suited to the particular use contemplated.

Unless otherwise noted, the terms “a” or “an,” as used in the specification and claims, are to be construed as meaning “at least one of” In addition, for ease of use, the words “including” and “having,” as used in the specification and claims, are inter-changeable with and have the same meaning as the word “comprising.” In addition, the term “based on” as used in the specification and the claims is to be construed as meaning “based at least upon.”

What is claimed is:

1. A method for object characterization and authentication, comprising:
establishing, over a communication network, a connection between a network accessible image capturing device and a remote computing device;
capturing a first image of an object using the image capturing device before performing an action in relation to the object;
identifying a distinguishing feature of the object on the first image of the object; capturing a second image of the object using the image capturing device after performing the action in relation to the object; and
identifying the distinguishing feature of the object on the second image of the object.

2. The method of claim 1, the image capturing device comprising one or more web services, the one or more web services including a device user interface accessible by the remote computing device via the connection.

3. The method of claim 2, the capturing of the first image comprising sending a first image capture command from the remote computing device to the image capturing device via the device user interface, and the capturing of the second image comprising sending a second image capture command from the remote computing device to the image capturing device via the device user interface.

4. The method of claim 2, comprising:
accessing at least one of the first and second images of the object on the remote computing device via the device user interface.

5. The method of claim 1, comprising:
marking, via the device user interface, the identified distinguishing feature of the object on the first image of the object; and
annotating at least one of owner information and object information on the first image of the object.

6. The method of claim 5, comprising:
generating a first communication that includes the first captured image of the object with the distinguishing feature marked.

7. The method of claim 5, comprising:
marking, via the device user interface, the distinguishing feature of the object on the second image of the object.

8. The method of claim 7, comprising:
generating a second communication that includes both the first captured image of the object with the distinguishing feature marked, and the second captured image of the object with the distinguishing feature marked.

9. The method of claim 1, the object including a gemstone, the distinguishing feature of the object including at least one of a type of object, measurement of the object, a diameter of the object, an inclusion of the object, a rating or grading of the object, and an identifier on the object.

10. The method of claim 1, wherein the image capturing device includes a microscope.

11. A computing device configured for object characterization and authentication, comprising:
a processor;
memory in electronic communication with the processor, wherein the memory stores computer executable instructions that when executed by the processor cause the processor to perform the steps of:
establishing, over a communication network, a connection between a network accessible image capturing device and the computing device;
capturing a first image of an object using the image capturing device before performing an action in relation to the object;
identifying a distinguishing feature of the object on the first image of the object;
capturing a second image of the object using the image capturing device after performing the action in relation to the object; and
identifying the distinguishing feature of the object on the second image of the object.

12. The computing device of claim 11, the image capturing device comprising one or more web services, the one or more web services including a device user interface accessible by the computing device via the connection.

13. The computing device of claim 12, the capturing of the first image comprising sending a first image capture command from the computing device to the image capturing device via the device user interface, and the capturing of the second image comprising sending a second image capture command from the computing device to the image capturing device via the device user interface.

14. The computing device of claim 12, wherein the instructions executed by the processor cause the processor to perform the steps of:
accessing at least one of the first and second images of the object on the computing device via the device user interface.

15. The computing device of claim 11, wherein the instructions executed by the processor cause the processor to perform the steps of:
marking, via the device user interface, the identified distinguishing feature of the object on the first image of the object; and
annotating at least one of owner information and object information on the first image of the object.

16. The computing device of claim 15, wherein the instructions executed by the processor cause the processor to perform the steps of:
generating a first communication that includes the first captured image of the object with the distinguishing feature marked.

17. The computing device of claim 15, wherein the instructions executed by the processor cause the processor to perform the steps of:
marking, via the device user interface, the distinguishing feature of the object on the second image of the object.

18. The computing device of claim 17, wherein the instructions executed by the processor cause the processor to perform the steps of:
generating a second communication that includes both the first captured image of the object with the distinguishing feature marked, and the second captured image of the object with the distinguishing feature marked.

19. A non-transitory computer-readable storage medium storing computer executable instructions that when executed by a processor cause the processor to perform the steps of:
establishing, over a communication network, a connection between a network accessible image capturing device and a remote computing device;
capturing a first image of an object using the image capturing device before performing an action in relation to the object;
identifying a distinguishing feature of the object on the first image of the object; capturing a second image of the object using the microscope after performing the action in relation to the object; and
identifying the distinguishing feature of the object on the second image of the object.

20. The computer-program product of claim 19, the image capturing device comprising one or more web services, the one or more web services including a device user interface accessible by the remote computing device via the connection.
